(19) United States
(12) Patent Application Publication
(10) Pub. No.: US 2023/0330384 A1
(43) Pub. Date: Oct. 19, 2023

(54) Method of delivering an olfactory composition and system for delivering such an olfactory composition

(71) Applicant: Enzo Didi, San Francisco, CA (US)

(72) Inventor: Enzo Didi, San Francisco, CA (US)

(21) Appl. No.: 18/340,157

(22) Filed: Jun. 23, 2023

(51) Int. Cl. A61M 21/00; G16H 20/70; G16H 10/20; A61B 5/16

(52) U.S. Cl. CPC A61M 21/00; G16H 20/70; G16H 10/20; A61B 5/165; A61M 2021/0016; A61M 2021/0027; A61M 2021/0044

(57) ABSTRACT

The invention relates to a method of delivering an olfactory composition, the method comprising the following steps: A1. retrieving at least one index of the physiological and/or emotional state of at least one user; A2. determining a physiological and/or emotional state from the index of the physiological and/or emotional state of said user; A3. determining at least one olfactory composition based at least in part on the determined physiological and/or emotional state of the user, A4. issuing said at least one determined olfactory composition. The invention also relates to a system for delivering such a suitable olfactory composition.

### TECHNICAL AREA

The invention relates to the field of providing olfactory ambience in living spaces.

The invention relates more particularly to a method of delivering an olfactory composition and a system for delivering such an olfactory composition.

### STATE OF THE ART

It's well known that scents and other olfactory stimuli play a role in the moods of those exposed to them. Chamomile essential oils, for example, are known to reduce anxiety, not least because of their high flavonoid content.

In order to exploit such a role, it is known in the prior art to use scent diffusers, based on sprays, which enable users to ambience their interiors by spraying a given olfactory composition. However, while such diffusers can provide an olfactory stimulus throughout the day that can potentially be tailored to the user, this is not necessarily the case. When this is not the case, adaptation of this stimulus requires voluntary intervention by the user to change the olfactory composition sprayed. What's more, whatever the choice of new olfactory composition, the user is not always in a position to determine which composition would be suitable for him or her.

Thus, there would be an interest in providing a process for supplying an olfactory composition adapted to the user in an at least partially automatic way, in order to provide him with an olfactory ambiance that is perfectly adapted to him.

### EXPLANATION OF THE INVENTION

The invention aims to remedy the above problem and thus has the object of providing a process adapted to provide the emission of a composition that is perfectly suited to a user of said process.

To this end, the invention relates to a method of delivering an olfactory composition, the method comprising the following steps: A1. retrieving at least one index of the physiological and/or emotional state of at least one user, A2. determining a physiological and/or emotional state from the index of the physiological and/or emotional state of said user, A3. determining at least one olfactory composition based at least in part on the determined user's physiological and/or emotional state, A4. issuing said at least one determined olfactory composition.

Such a process enables the emission of an optimal composition to ambience the interior of the user's home, without the user having to make a choice. In fact, the invention's process makes it possible to determine the olfactory composition(s) best suited to the user, without the latter having to intervene. What's more, with a suitable device, this emission does not require any intervention on the part of the user. Indeed, as is possible within the scope of the invention, the emission device can feature a plurality of distinct components to enable fine-tuning of the olfactory composition to the user's physiological and/or emotional state.

Step A1. retrieving the user's physiological and/or emotional state index comprises the following sub-steps: A11. submitting a questionnaire to the user, said questionnaire including at least one question, A12. retrieving the user's response(s) to said questionnaire, the or each of said responses forming an index of the user's physiological and/or emotional state.

This type of questionnaire provides precise information about the user's physiological and/or emotional state, since it is possible to question the user directly about his or her feelings. In this way, the physiological and/or emotional state, and therefore the at least one olfactory composition based on it, can be finely determined. As a result, the olfactory composition can be particularly tailored.

The step A1. of retrieving the physiological and/or emotional state index may comprise the following sub-step: A13. retrieving at least one physiological quantity of the user from a connected object belonging to the user, said physiological quantity forming a physiological and/or emotional state index of the user.

Such a magnitude provides an objective measure of the user's physiological and/or emotional state. Indeed, this type of object, such as a connected watch, a connected scale or a connected sleep sensor, provides access to the user's physiological data, which may also reflect the user's emotional state. Thus, it is possible to determine with such a user physiological and/or emotional state index, a physiological and/or emotional state of the user without any real user intervention. The olfactory composition can thus adapt to changes in the user's physiological and/or emotional state without the user having to take any action.

The physiological and/or emotional state index retrieval step may comprise the following sub-step: A14. retrieving information relating to the user's use of at least one IT tool, such as a cell phone, computer or voice assistant, said information each forming an index of the user's physiological and/or emotional state.

The uses made of IT tools by users, such as their choice of music, the websites they visit, the Internet searches they carry out or the posts they make on social networks, will necessarily reflect their emotional state and, by extension, their physiological and/or emotional state. So, with the user's consent, it is possible to obtain precise clues as to the user's emotional state, or even physiological state, without the user having to intervene.

Step A3. of determining an olfactory composition based at least in part on the physiological and/or emotional state of the determined user comprises determining a respective proportion between at least two compounds intended to participate in the composition emitted as a function of the physiological and/or emotional state of the determined user, step A4. of emitting the determined olfactory composition comprising emitting each of these at least two compounds according to their respective proportion so as to form the olfactory composition adapted to the physiological and/or emotional state of the determined user.

The use of at least two, preferably three, compounds in this way makes it easy to adapt the composition of compounds in the composition, which can be achieved by changing the proportion of each of the at least two compounds.

The step A3. of determining an olfactory composition based at least in part on the physiological and/or emotional state of the user determined can enable the determination of at least two adapted olfactory compositions, said step A3. of determining an olfactory composition comprising a sub-step of selection by the user of the adapted olfactory composition to be emitted from said at least two adapted olfactory compositions.

In this way, the user can choose an olfactory composition that is adapted to his physiological and/or emotional state and perfectly suited to his tastes.

During step A4. of emitting an olfactory composition adapted to the physiological and/or emotional state of the specific user, a sound and/or light stimulus adapted to the physiological and/or emotional state of the specific user is also emitted in association with the emission of the adapted olfactory composition.

An additional stimulus of this kind, complementary to the emission of the olfactory composition, makes it possible to perfect the adaptation of the olfactory ambience enabled by the invention.

The invention further relates to a system for delivering an olfactory composition comprising: —a control device able to retrieve at least one index of the physiological and/or emotional state of at least one user, and an adapted olfactory composition delivery device adapted to deliver said adapted olfactory composition, wherein the control device and the emission device are configured to determine a physiological and/or emotional state from the index of the physiological and/or emotional state of said user, and to determine the adapted olfactory composition based at least in part on the determined physiological and/or emotional state of the user.

Such a system makes it possible to implement a process according to the invention and to benefit from the advantages associated with it.

This can be the control device which is configured to determine a physiological and/or emotional state from the index of the physiological and/or emotional state of said user, the control device being a computing tool, such as a cell phone, computer or voice assistant.

A computerized tool such as this enables the physiological and/or emotional state to be determined in a way that can easily be adapted to the needs and evolutions of the system. In this way, it is easy to optimize the determination process according to the changes to be made to the system.

The emission device may comprise: —at least two housings each intended to receive a respective capsule containing a possible compound of said olfactory composition preferably in the form of solid granules, each capsule having an outlet face comprising openings in order to allow diffusion of the compound, —for each housing, a variable diffusion generating system in order to generate a variable diffusion of the compound through the outlet face of the capsule housed in said housing for each housing, a variable diffusion generation system to generate a variable diffusion of the compound through the outlet face of the capsule housed in said housing; the emission device being further configured to vary the diffusion of compound generated by each of the variable diffusion generation systems in order to vary the proportion of compounds in the olfactory composition emitted by the emission device.

Thanks to the possibility of arranging at least two capsules, such an emission device enables easy adaptation of the olfactory composition. As a result, this device is particularly suitable for implementing a process according to the invention.

According to a possibility compatible with the invention but not limited to it, there is provided a method of emitting an olfactory composition, the olfactory composition comprising at least one first compound of a type selected from a top note type compound and a middle note type compound, and at least one second compound of a type selected from a middle note type compound and a base note type compound, the first and second compounds being compounds of a distinct type, the process comprising a step of emitting the olfactory composition with a variation of the respective composition in the first and second compounds over time to take account of the difference in volatility between the first and second compounds.

Thus, as the first compound has a higher volatility, the olfactory composition can, in a first step, have a given composition, such as that determined according to the process of the present invention, and, in a second step, have a composition in which the proportion of the first compound is increased to compensate for its higher volatility relative to that of the second compound.

Similarly, according to this possibility compatible with the invention, the olfactory composition can comprise at least the first compound of a top note type, the second compound of a middle note type and a third compound of a base note type, during the step of emitting the olfactory composition, the olfactory composition presents a variation in the respective composition of the first, second and third compounds over time to take account of the difference in volatility between the first, second and third compounds.

In the same way, since the first compound has a higher volatility than the second compound, which in turn has a higher volatility than the third compound, this difference in volatility can be corrected throughout the emission of the composition. In this way, the olfactory composition can initially have a given composition, then in a second step, the composition is corrected to increase the relative proportion of the first compound, and in a third step, the composition can be corrected to increase the relative proportion of the second compound.

In this way, it is possible to perfect the olfactory ambience so that it is little or not at all affected by the difference in volatility between the different compounds.

### BRIEF DESCRIPTION OF THE DRAWINGS

The present invention will be better understood on reading the description of exemplary embodiments, given purely by way of illustration and in no way limiting, with reference to the appended drawings on which:

FIG. 1A illustrates a system according to the invention.

FIG. 1B illustrates an olfactory composition delivery device according to the invention with the cover removed.

FIG. 2A illustrates a mobile terminal of a system according to the invention, showing the menu for implementing the process according to the invention.

FIG. 2B illustrates the same mobile terminal displaying a first questionnaire used as part of the olfactory composition emission process according to the invention.

FIG. 2C illustrates the same mobile terminal displaying a second questionnaire used as part of the olfactory composition emission process according to the invention.

FIG. 2D illustrates the result of determining two olfactory compositions based at least in part on the user's physiological and/or emotional state determined from the first and second questionnaires.

FIG. 3 illustrates examples of physiological and/or emotional states that can be determined using a process according to the invention.

FIG. 4A shows a flowchart of the process according to the invention.

FIG. 4B illustrates a flowchart specific to the step of determining the user's physiological and/or emotional state.

Identical, similar or equivalent parts of the various figures bear the same numerical references, so that it is easier to move from one figure to another.

The various parts shown on the figures are not necessarily shown on a uniform scale, to make the figures easier to read.

The various possibilities (variants and realization modes) should be understood as not being mutually exclusive and can be combined with each other.

### A DETAILED DESCRIPTION OF SPECIFIC PRODUCTION METHODS

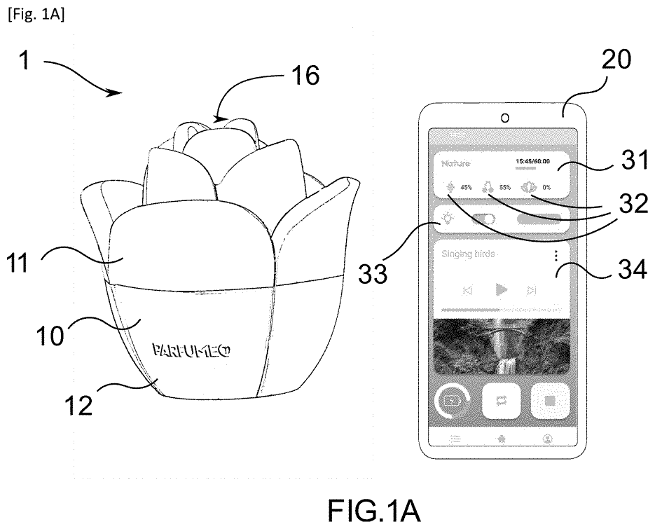

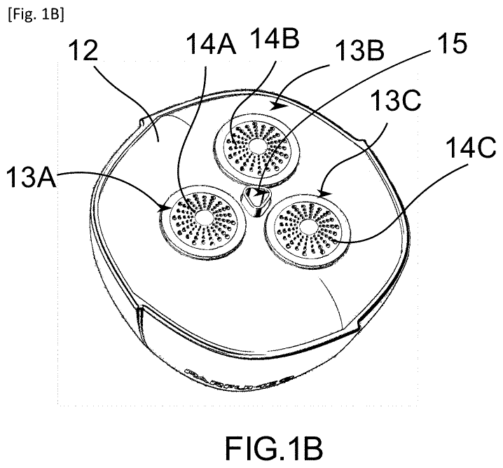

FIG. 1A illustrates an adapted olfactory composition delivery system 1 capable of emitting said adapted olfactory composition as part of a process according to the invention.

While such a delivery system 1 is particularly suitable for providing a pleasant atmosphere inside a user's home, the invention is not limited to this use. For example, such a system can be fitted to a user's vehicle or business premises without departing from the scope of the invention.

It should be noted that in the present embodiment, as shown in FIG. 1A, the delivery system 1 more precisely comprises an olfactory composition emission device 10 and a mobile terminal 20 of the cell phone type, or more precisely of the smartphone type.

FIG. 1A shows that the emission device 10 comprises a lid 11 and a body 12 which is surmounted by said lid 11. The cover has an opening 16 through which the olfactory composition is emitted.

As shown in FIG. 1B, the olfactory composition delivery device 10, and more precisely its body 12, has three housings 13A, 13B, 13C in which are arranged three capsules 14A, 14B, 14C, each containing a respective compound likely to be part of said olfactory composition. In each of the capsules 14A, 14B, 14C, any compound of said olfactory composition may be provided in the form of solid granules. Each capsule has an inlet face, not referenced, and an outlet face, not shown, both having openings to define an air flow path between the inlet face and the outlet face. The flow path passes through said granules in order to extract therefrom the compound of the olfactory composition, this compound being chosen as volatile to allow its emission into an air stream.

The emission device 10 further comprises, for each housing 13A, 13B, 13C a variable airflow generation system, not shown, to generate a variable airflow at said housing along the airflow path. Such a variable airflow generation system can be provided by a fan arranged opposite one end of the housing which corresponds to the outlet face of the capsule 14A, 14B, 14C housed therein. Air flow variability can thus be obtained by modifying the fan's rotational speed. It should be noted that in the present embodiment, each fan is arranged in suction mode, the airflow it generates being obtained by sucking air through the corresponding capsule 14A, 14B, 14C and its inlet face. It should be noted that to obtain the composition, the air flow generated by each of the fans is combined in a common duct 15, which is itself in fluid communication with an outlet.

Such an airflow generation system forms a variable diffusion generation system capable of enabling variable diffusion of the corresponding capsule compound.

Alternatively, the variable diffusion generation system can include a heating resistor capable of applying heat to the capsule and the compound contained therein in such a way as to promote diffusion of the compound. Thus, according to this variant, with the heat applied by each resistor, it will be possible to promote or not the diffusion/emission of each of the compounds and thus modify the compound composition of the olfactory composition. Of course, such a heating resistor can be provided on its own or in conjunction with a fan (this fan can be dedicated to one dwelling or shared between several dwellings, or even all the dwellings). It should be noted that in the context of such a possibility, the compound in each capsule 14A, 14B, 14C can easily take a form other than that of solid granules.

According to another variant, the system for generating variable diffusion of the compound can include a system for supplying a heating resistor directly integrated into the corresponding capsule. According to this other variant, the capsule integrates such a heating resistor so as to be as close as possible to the compound.

In this way, by modulating the speed of each of the fans, the emission device 10 can modulate the proportion of each of the compounds in the olfactory composition and thus provide an adaptation of the latter. The emission device 10 further comprises a control circuit, not shown, configured to control the individual fans as a function of the composition of compounds in the olfactory composition. The control circuit is also adapted, in the context of the present embodiment, to be connected from a wireless connection, such as for example a Wifi™ connection or a Bluetooth™ connection, to the mobile terminal.

It will be noted that, in the context of the present embodiment and particularly advantageously, the emission device 10 may also comprise an illumination system, not shown, such as one or more light-emitting diodes adapted to provide illumination light of variable color and/or intensity and a music diffusion system, not shown, such as a loudspeaker. According to this possibility, the control circuit is adapted to control said lighting and music diffusion systems.

In the present embodiment, the mobile terminal 20, acting as a control device, is able to retrieve at least one index of the physiological and/or emotional state of at least one user, and the device for emitting an adapted olfactory composition 10 is able to emit said adapted olfactory composition. The mobile terminal 20 and the emission device 10 are configured to determine a physiological and/or emotional state from the index of the physiological and/or emotional state of said user, and to determine the adapted olfactory composition based at least in part on the determined physiological and/or emotional state of the user.

More specifically, in the present embodiment, it is the mobile terminal 20 that is adapted to determine a physiological and/or emotional state and to determine the appropriate olfactory composition.

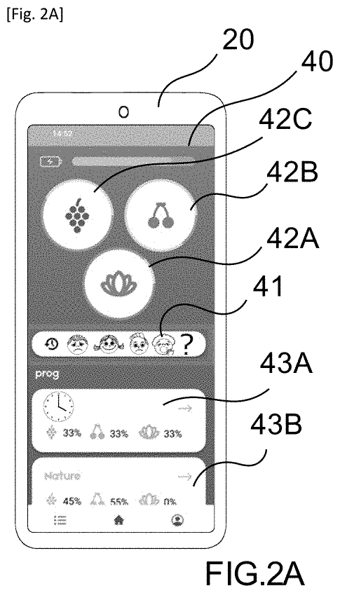

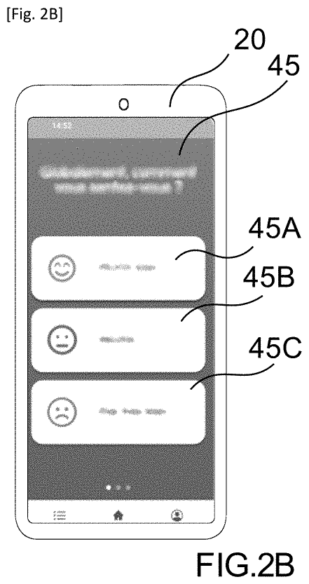

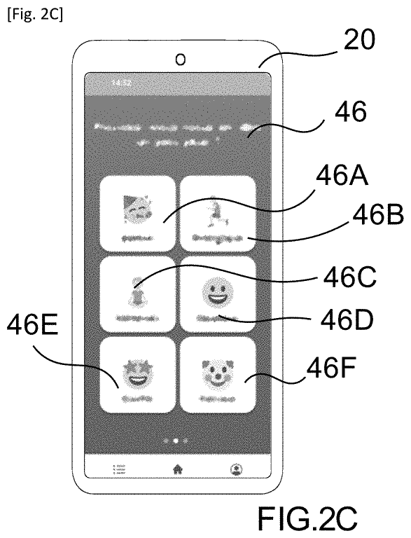

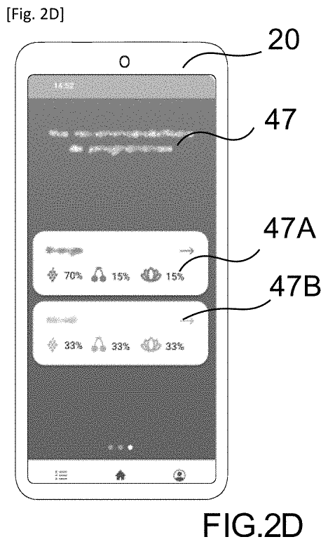

As shown in FIG. 1A and FIGS. 2A to 2D, such adaptation is provided via a dedicated application installed on the mobile terminal 20. As can be seen in FIG. 1A, the application can feature various buttons, including, for example, a button for emitting 31 an olfactory composition, in this case a composition made up of 45% of a compound 32 corresponding to grapes, 55% of a compound 32 corresponding to cherries, and 0% of a compound 32 corresponding to lotus, a button for controlling the lighting system 33 of the emission device 10, and a button for controlling the music diffusion system 34 in order to play a song through the music diffusion system.

With such a dedicated application, as shown in FIG. 2A, the mobile terminal 20 is also configured to present, for example as part of an application home page 40, a “mood determination” section 41 represented by a number of physiological and/or emotional states and a question mark. Note that this home page example also gives access to individual diffusion buttons 42A, 42B, 42C for each of the capsule compounds fitted to the diffusion device, and to two buttons for the last two programs 43A, 43B used. As can be seen from this example, the capsules fitted to the broadcast device 10 are capsules comprising a lotus-based compound, a cherry-based compound and a grape-based compound respectively.

When the user selects the “mood determination” section (41), he or she is presented with two pages of questionnaires (45, 46), as shown in FIGS. 2B and 2C. The first, simplified questionnaire 45 asks the user how he or she feels in general, and the user can respond by selecting the appropriate emoticon: “rather good” 45A corresponding to the smiling emoticon, “neutral” 45B corresponding to the neutral emoticon and “not very good” 45C corresponding to the sad emoticon. The second, more specific questionnaire appears according to the answer given to the first questionnaire. The example shown in FIG. 2C corresponds to a “rather well” response (smiling emoticon corresponding to reference 45A) given by the user to the first questionnaire, and a request is made for clarification, for example by means of a “can you tell us more” question, regarding the user's condition. A user who has indicated that he or she is “quite well” can choose between six propositions: “happy” 46A, represented by a “party” emoticon; “energetic” 46B represented by a “runner” emoticon; “relaxed” 46C, represented by a “yoga” emoticon; “happy” 46D, represented by a “smiling” emoticon; “excited” 46E represented by a “stars in the eyes” emoticon; and “prankster” 46F represented by a “clown” emoticon.

The answers given by the user to these two questionnaires provide clues as to the user's physiological and/or emotional state.

Of course, if the choices are represented by “emoticons”, such representation is not necessary, and another type of representation, such as a photo or drawing, can be used as an alternative.

It should also be noted that it is also possible, as an alternative or in addition to such multiple-choice questionnaires, to provide one or more open-ended questions, with the user having to express his or her response in his or her own words, either in writing (e.g. using a virtual keyboard) or orally (e.g. using a microphone on the mobile terminal 20). In this way, the application can directly ask, by text or vocalization, “how the user feels”. Such responses to this open-ended question(s) also form a clue to the user's physiological and/or emotional state.

Once the indication(s) of the user's physiological and/or emotional state have been obtained, the same application is able to determine a physiological and/or emotional state of the user. From the determined physiological and/or emotional state, the application is able to determine at least one olfactory composition based at least in part on the determined physiological and/or emotional state of the user. Here, as shown in FIG. 2D, such a determination has enabled the application to determine two different olfactory compositions 47A, 47B adapted to the user's physiological and/or emotional state: a first qualified as “Energy” comprising 70% grape-based compound, 15% cherry-based compound and 15% lotus-based compound; and a second qualified as “Awakening” comprising 33.33% grape-based compound, 33.33% cherry-based compound and 33.33% lotus-based compound. When several different olfactory compositions 47A, 47B are determined, the olfactory compositions determined can be presented to the user so that he/she selects the one he/she considers most suitable, via an adapted 47 menu.

Of course, if the answers to these questions/questionnaires can provide indices of the user's physiological and/or emotional state, other indices of physiological and/or emotional state can be used in addition to or as an alternative to these answers without departing from the scope of the invention. Thus, within the scope of the invention, it is also conceivable to retrieve at least one physiological quantity of the user from a connected object belonging to the user, said physiological quantity forming an index of the user's physiological and/or emotional state. According to this possibility, such a connected object can be, for example and in a non-limiting way, a connected watch (also known under the English denomination “smartwatch”), a connected bracelet or more generally an activity sensor, a connected scale, or even a connected sleep sensor. Similarly, the invention can also be used to retrieve information relating to the user's use of at least one IT tool, such as a cell phone, computer or voice assistant, said information each forming an index of the user's physiological and/or emotional state. Indeed, if we take the example of the cell phone, the Internet searches made by the user and the music he/she listens to are particularly relevant with regard to the user's physiological and/or emotional state. If the user gives access to this data, the use of a questionnaire or a question put to the user concerning his/her physiological and/or emotional state is no longer necessarily necessary.

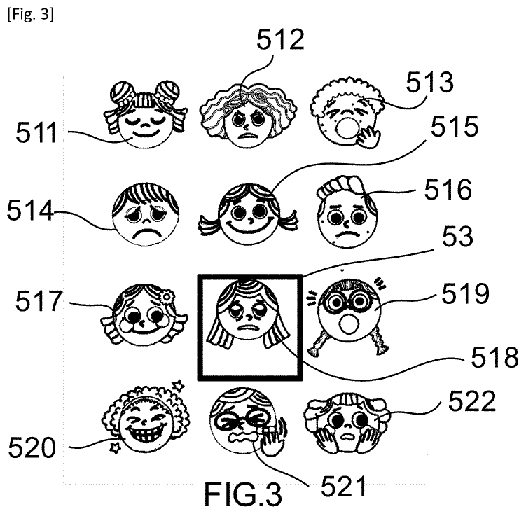

From all these indications of the user's physiological and/or emotional state, it is therefore possible to determine the user's physiological and/or emotional state. FIG. 3 shows an example of a list of states that can be determined within the framework of the invention. Such a list, where each state is represented by a drawing, includes:
—511 calm; —512 angry; —513 sleepy; —514 sad; —515 happy; —516 worried; —517 shy; —518 tired; —519 shocked; —520 proud; —521 hurt; —522 afraid.

Of course, such a list is provided by way of example only and can be adapted according to the type of olfactory compositions that can be emitted by the emission device 10. It will be noted that, by way of example, in the case illustrated in FIG. 3, the physiological and/or emotional state of the user detected 53 is a tired state.

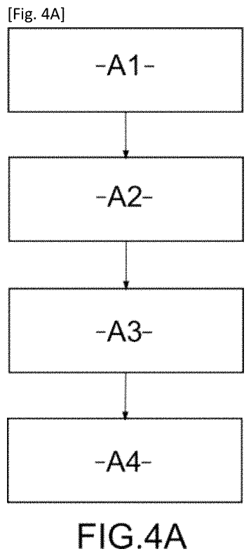

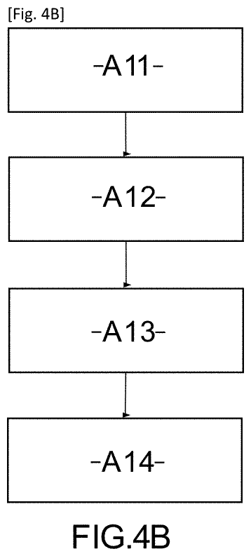

Such a delivery system 1 comprising a delivery device 10 according to the invention and a mobile terminal 20 can be adapted to enable the implementation of a process for delivering a suitable olfactory composition comprising, as shown in FIG. 4A, the following steps: A1. Retrieval of at least one index of the physiological and/or emotional state of at least one user, A2. Determination of a physiological and/or emotional state from the index of the physiological and/or emotional state of said user, A3. Determination of at least one olfactory composition based at least in part on the physiological and/or emotional state of the user determined, A4. Emission of said at least one determined olfactory composition.

Step A1. for retrieving at least one index of the user's physiological and/or emotional state may comprise, as shown in FIG. 4B, the following sub-steps: A11. Submitting a questionnaire to the user, said questionnaire including at least one question, A12. Retrieving the user's response(s) to said questionnaire, the or each said response forming an index of the user's physiological and/or emotional state.

In addition to or instead of such sub-steps, step A1. for retrieving at least one index of the user's physiological and/or emotional state may also comprise, as shown in FIG. 4B: A13. Recovering at least one physiological quantity of the user from a connected object belonging to the user, said physiological quantity forming an index of the user's physiological and/or emotional state, and/or A14. Retrieval of information relating to the user's use of at least one IT tool, such as a cell phone, computer or voice assistant, said information each forming an index of the user's physiological and/or emotional state.

In step A3. of determining an olfactory composition based at least in part on the physiological and/or emotional state of the user determined, at least two suitable olfactory compositions may be determined. According to this possibility, step A3. of determining an olfactory composition may comprise a sub-step of selection by the user of the adapted olfactory composition to be emitted from said at least two adapted olfactory compositions.

Of course, if the olfactory composition emitted by the emission device can be based on the physiological and/or emotional state of the user, it can also be based on other parameters such as the time of day, the room temperature and the weather. In addition to the user's physiological and/or emotional state, the olfactory composition can also be adapted in the morning to promote waking and in the evening to promote falling asleep.

According to this possibility, it is also conceivable that the delivery system 1 could, as a complement to the implementation of the process according to the invention, enable the user, if he so wishes, to adapt the olfactory composition according to his desire. In this way, the application can also present the user with pre-programmed olfactory compositions that he or she can choose from in order to select the olfactory ambience for the interior of his or her home.

It should be noted that, in the present embodiment, it is the application installed on the mobile terminal that is used to determine the user's physiological and/or emotional state and the appropriate olfactory composition. It is also conceivable, without departing from the scope of the invention, that one or both of these steps be implemented by the transmitter device 10. Alternatively, the mobile terminal 20 application can be connected to a server, and the mobile terminal can determine the user's physiological and/or emotional state and/or the appropriate olfactory composition by cooperating with said server.

In addition to the description of the process according to the invention and the various means of implementing it described above, according to a possibility compatible with the invention but not limited to it, it is possible to provide a process for emitting an olfactory composition in order to compensate for the difference in volatility of the various compounds that make it up.

Thus, according to this possibility compatible with the invention, a first capsule 14A may comprise a top note type compound, a second capsule 14B a heart note type compound and a third capsule 14C a base note type compound. This emission process may comprise a step of emitting the olfactory composition in which the olfactory composition presents a variation in the respective composition of the first, second and third compounds over time to take account of the difference in volatility between the first, second and third compounds.

According to this possibility compatible with the invention, the first compound therefore has a higher volatility than the second compound, which in turn has a higher volatility than the third compound. It is thus possible to compensate for this difference in volatility throughout the emission of the olfactory composition. In this way, the olfactory composition can initially have a given composition, in a second step, the composition is corrected to increase the relative proportion of the first compound, and in a third step, the composition can be corrected to increase the relative proportion of the second compound.

Of course, if within the scope of the present presentation of this possibility compatible with the invention, a top note type compound, a heart note type compound and a base note type compound are provided, such a possibility compatible with the inventor covers other configurations without going outside the scope of this possibility. For example, two top note compounds and one base note compound may be provided, whereby the olfactory composition can be adapted over time to compensate for the greater volatility of the two top note compounds in relation to the base note compound. This is particularly advantageous when one compound is more volatile than another in the olfactory composition.

1. A method of delivering an olfactory composition, the method comprising the following steps:
A1. Recovery of at least one index of the physiological and/or emotional state of at least one user,
A2. Determination of a physiological and/or emotional state from said user's physiological and/or emotional state index,
A3. Determination of at least one olfactory composition based at least in part on the physiological and/or emotional state of the determined user,
A4. Emission of said at least one olfactory composition.

2. A method of delivering an olfactory composition according to claim 1 wherein step A1. of retrieving the user's physiological and/or emotional state index comprises the following sub-steps:
A11. Submission of a questionnaire (45, 46) to the user, said questionnaire (45, 46) including at least one question, A12. Retrieval of the user's response(s) (45A, 45B, 45C, 46A, 46B, 46C, 46D, 46E, 46F) to said questionnaire, the or each of said responses forming an index of the user's physiological and/or emotional state.

3. A method of delivering an olfactory composition according to claim 1 or 2, wherein step A1. of retrieving the physiological and/or emotional state index comprises the following sub-step:
A13. Recovering at least one physiological parameter of the user from a connected object belonging to the user, said physiological parameter forming an index of the user's physiological and/or emotional state.

4. A method of delivering an olfactory composition according to any one of claims 1 to 3, wherein the step of retrieving the physiological and/or emotional state index comprises the substep:
A14. Recovering information relating to the user's use of at least one IT tool, such as a cell phone, computer or voice assistant, said information each forming an index of the user's physiological and/or emotional state.

5. A method of delivering an olfactory composition according to any one of claims 1 to 4, wherein step A3. of determining an olfactory composition based at least in part on the physiological and/or emotional state of the user determined comprises determining a respective proportion between at least two compounds intended to participate in the composition emitted as a function of the physiological and/or emotional state of the user determined,
step A4. of emitting the determined olfactory composition comprising the emission of each of these at least two compounds in their respective proportion so as to form the olfactory composition adapted to the physiological and/or emotional state of the determined user.

6. A method of dispensing an olfactory composition according to claims 1 to 5, wherein step A3. of determining an olfactory composition based at least in part on the physiological and/or emotional state of the user determined enables the determination of at least two suitable olfactory compositions, said step A3. of determining an olfactory composition comprising a sub-step of selection by the user of the suitable olfactory composition to be dispensed from said at least two suitable olfactory compositions.

7. A method of delivering an olfactory composition according to any one of claims 1 to 6, wherein in step A4. of emitting an olfactory composition adapted to the physiological and/or emotional state of the particular user, there is also provided the emission of a sound and/or light stimulus adapted to the physiological and/or emotional state of the particular user and in association with the emission of the adapted olfactory composition.

8. An olfactory composition delivery system comprising:
a control device able to retrieve at least one index of the physiological and/or emotional state of at least one user and,
a device for emitting an adapted olfactory composition capable of emitting said adapted olfactory composition,
wherein the control device and the emission device are configured to determine a physiological and/or emotional state from the index of the physiological and/or emotional state of said user, and to determine the adapted olfactory composition based at least in part on the determined physiological and/or emotional state of the user.

9. A system for delivering an olfactory composition according to claim 8 wherein it is the control device that is configured to determine a physiological and/or emotional state from the index of the physiological and/or emotional state of said user, the control device being a computer tool, such as a cell phone, computer or voice assistant.

10. A system for delivering an olfactory composition according to claim 8 or 9, wherein the delivery device comprises:
at least two housings (13A, 13B, 13C) each designed to receive a respective capsule (14A, 14B, 14C) containing a possible compound of said olfactory composition, preferably in the form of solid granules, each capsule (14A, 14B, 14C) having an outlet face comprising openings to allow diffusion of the compound,
for each housing (13A, 13B, 13C), a variable diffusion generation system in order to generate a variable diffusion of the compound through the outlet face of the capsule housed in said housing;
the emission device (10) being further configured to vary the compound diffusion generated by each of the variable diffusion generation systems in order to vary the proportion of compounds in the olfactory composition emitted by the emission device (10).
